(19) United States
(12) Patent Application Publication
(10) Pub. No.: US 2002/0094575 A1
(43) Pub. Date: Jul. 18, 2002

(54) Compositions and methods for stable transformation using Mu bacteriophage cleaved donor complex

(75) Inventor: Hideki Suzuki, Fremont, CA (US)

Correspondence Address: ALSTON & BIRD LLP, PIONEER HI-BRED INTERNATIONAL, INC., BANK OF AMERICA PLAZA, 101 SOUTH TYRON STREET, SUITE 4000, CHARLOTTE, NC, 28280-4000, US

(73) Assignee: Pioneer Hi-Bred International, Inc.

(21) Appl. No.: 09/951,829

(22) Filed: Sep. 13, 2001

Related U.S. Application Data
(60) Provisional application No. 60/232,979, filed on Sep. 14, 2000.

(51) Int. Cl. C12N 15/74; C12N 15/87

(52) U.S. Cl. 435/455; 435/320.1

(57) ABSTRACT

Compositions and methods for stably integrating a nucleotide sequence of interest into the genome of an organism are provided. The compositions are novel integration vectors comprising an active cleaved donor complex, or CDC, which comprises a Mu transposable cassette of a mini-Mu plasmid or precleaved mini-Mu plasmid. The Mu transposable cassette comprises the nucleotide sequence of interest and is bound to MuA transposase to form the CDC. In the presence of MuB-bound host DNA, the MuA protein bound as part of the MuA tetrameric core facilitates transfer of the Mu transposable cassette, which comprises the nucleotide sequence of interest, into the host DNA at the site to which MuB is bound. The inserted Mu transposable cassette remains stably integrated at this site within the host genome. Methods of the invention include transforming a host organism with such an integration vector and with a plasmid comprising the MuB coding sequence operably linked to a promoter that drives expression in the host organism. Transient expression of the MuB accessory protein results in random binding of this protein to the genome of the organism. MuB-bound genomic DNA becomes the site for insertion of the integration vectors of the invention and stable integration of the nucleotide sequence of interest within the organism's genome. Transformed plant cells, tissues, plants, and seed are also provided.

### FIELD OF THE INVENTION

The invention relates to the field of genetic engineering, specifically to integration vectors and their use in stable transformation of organisms.

### BACKGROUND OF THE INVENTION

Genetic modification techniques enable one to insert exogenous nucleotide sequences into an organism's genome to alter the phenotype of the organism. Depending upon the desired outcome of the modification, manipulation of a genome may be aimed at creating, enhancing, decreasing, or even disrupting the production of a functional gene product.

A number of methods have been described and utilized to produce stably transformed prokaryotic and eukaryotic cells. All of these methods are based on introducing a foreign DNA into a host cell and subsequent isolation of those host cells containing the foreign DNA integrated into the genome. These methods generally rely upon cellular recombination events that utilize host proteins to achieve random integration of the foreign DNA. Efficiency of transformation is dependent upon the organism and transformation method.

In plant species, for example, methods of transformation include the use of disarmed Agrobacterium species as well as microprojectile bombardment. Agrobacterium are relatively benign natural pathogens of dicotyledonous plants, and Agrobacterium-mediated transformation has been directed to both dicotyledonous and monocotyledonous species. The Agrobacterium species actively mediate transformation events as a part of the natural process of infecting a plant cell. However, the successful and reliable use of this method still tends to depend on the genotype of the plants and Agrobacterium used as well as the culture media used in the transformation process. Even under good conditions, the frequency of transformation is relatively low in some species.

Microprojectile bombardment entails bombardment of plant cells with dense microparticles carrying genetic material such as DNA sequences or plasmids. Microprojectile bombardment is less genotype-specific than Agrobacterium-mediated transformation. However, frequencies of stable transformation are also low with this method, due in part to an absence of natural mechanisms to mediate integration of the introduced genetic material into the plant genome.

Transposable genetic elements or transposons offer another means of genetically altering an organism. Transposons are DNA sequences that can move or transpose from one position to another position in a genome. These movable elements are found in a wide variety of prokaryotic and eukaryotic organisms. In vivo, intra-chromosomal transpositions as well as transpositions between chromosomal and non-chromosomal genetic material are known. In several systems, transposition is known to be under the control of a transposase enzyme that is typically encoded by the transposable element. The genetic structures and transposition mechanisms of various transposable elements are summarized, for example, in “Transposable Genetic Elements” in The Encyclopedia of Molecular Biology, ed. Kendrew and Lawrence (Blackwell Science, Ltd., Oxford, 1994), incorporated herein by reference.

Transposable elements normally comprise a gene encoding the transposase protein and a so-called transposable cassette, which comprises a resistance gene flanked by end sequences that are recognized by the transposase protein. Transposition of the transposable cassette into the genome of a host cell occurs via recognition of and interaction with the flanking end sequences of the transposable cassette by the transposase protein. Subsequent insertion into the genome of the host cell occurs randomly or at “hot spot” sites. In wild-type transposons, the transposase gene may reside within the transposable cassette. This provides a means for subsequent movement of the transposon following insertion within a host genome. For purposes of genetic engineering, further migration of an inserted transposon can be eliminated by repositioning the transposase gene outside the transposable cassette. Furthermore, the need for an active transposase protein, and in some instances additional accessory proteins, to assist with integration requires that the engineered host cell be provided with these accessory proteins, usually via transformation with the necessary genes encoding these proteins.

Unfortunately, despite improvements in transformation techniques and technology, the frequency of successful stable transformation events remains low. Thus, a continuing need exists for methods of transformation that enhance efficiency of DNA transfer and subsequent stable integration of the transferred DNA into the host organism's genome.

### SUMMARY OF THE INVENTION

Compositions and methods for stably integrating a nucleotide sequence of interest into the genome of an organism are provided. The compositions are integration vectors derived from the Mu bacteriophage cleaved donor complex (CDC). Cleaved donor complexes are obtained using a transposition reaction with a mini-Mu plasmid or precleaved mini-Mu plasmid as the transposon donor. The mini-Mu plasmid consists of two DNA regions: a Mu transposable cassette comprising a nucleotide sequence of interest and a non-Mu plasmid DNA domain. The non-Mu plasmid DNA domain may be removed following formation of the CDC. Where precleaved mini-Mu plasmids are the source, modifications to the non-Mu plasmid domain can occur prior to formation of the CDC. The resulting integration vectors of the invention comprise a Mu transposable cassette which further comprises nucleotide sequences of interest. MuA transposase is bound to the recognition elements in the transposable cassette so as to form a MuA tetrameric core and produce a cleaved donor complex. In the presence of MuB-bound host DNA, the MuA transposable cassette is inserted into the host DNA at the site to which MuB is bound. The inserted Mu transposable cassette remains stably integrated at this site within the host genome.

Methods of the invention utilize the novel integration vectors of the invention to genetically modify an organism's genome. The methods comprise engineering cells of the host organism so as to contain: 1) an integration vector of the invention; and 2) MuB protein. In some embodiments, MuB protein is produced in the host cell by a plasmid comprising the MuB gene operably linked to a promoter that drives expression in the host cell. In some embodiments, expression of MuB protein in the host cell is transient. Expression of the MuB accessory protein results in random binding of this protein to the genome of the organism. MuB-bound genomic DNA becomes a site for insertion of the integration vectors of the invention. Thus, the methods are useful for promoting stable integration of nucleotide sequences of interest into an organism's genome.

### BRIEF DESCRIPTION OF THE DRAWINGS

FIG. 1 schematically depicts the key features of the transposable region of the bacteriophage Mu, which serves as the basis for the key features of the wild-type mini-Mu plasmid. The Mu left end recognition sequence, which comprises the attL1 (L1), attL2 (L2), and attL3 (L3) end-type MuA transposase binding sites, the Mu right end recognition sequence, which comprises the attR1 (R1), attR2 (R2), and attR3 (R3) end-type MuA transposase binding sites, the transpositional enhancer sequence (also referred to herein as the internal activating sequence, or IAS), the MuA transposase sequence, and the MuB sequence are shown. In the wild-type mini-Mu plasmid, the transposable cassette region comprises the left end and right end recognition sequences flanking an internal nucleotide sequence which in turn comprises the IAS; this transposable cassette region is flanked by the non-Mu plasmid DNA domain (narrow solid black line).

FIG. 2 schematically depicts the normal molecular mechanism of Mu transposition. In the presence of MuB, intermolecular transposition occurs; in its absence, transposition is predominately intramolecular.

FIG. 3 schematically depicts Mu donor cleavage and strand transfer to a target site. L=left end recognition sequence; R=right end recognition sequence.

FIG. 4 schematically depicts formation of a Mu phage-derived cleaved donor complex (CDC) from a mini-Mu plasmid.

FIG. 5 schematically depicts formation of an active CDC integration vector of the invention from a precleaved mini-Mu plasmid.

FIGS. 6-8 schematically depict the MuB-directed process of integration of a cleaved donor complex. FIG. 6 schematically depicts binding of MuB to the host organism's genome following expression of MuB in the host cell.

FIG. 7 schematically depicts formation of a complex between genome-bound MuB and the integration vector.

FIG. 8 schematically depicts integration of the Mu transposable cassette region of the integration vector into the host organism's genome.

FIG. 9 shows a diagram of plasmid PC-4, a representative plasmid of the invention, which comprises DNA elements capable of forming a CDC complex: MuA binding sites R1, R2, and R3 positioned at the ends of a DNA fragment in inverted orientation. While PC-4 contains a selectable marker for use in bacteria, the use of a selectable marker is not essential to the present invention; in some embodiments, cotransformation of a selectable marker with a CDC and/or screening for transformants are performed. Other vectors of the present invention are shown in FIG. 10.

FIG. 10 shows a diagram of representative plasmids of the invention PC-1, PC-2, and PC-3. Each plasmid contains the MuA binding sites R1, R2 and R3 in inverted orientation.

### DETAILED DESCRIPTION OF THE INVENTION

The present invention is directed to compositions and methods for stable transformation of an organism with a nucleotide sequence of interest. By “stable” transformation is intended the nucleotide sequence of interest is integrated within the genome of the organism and is heritable in subsequent generations. Insertion of the nucleotide sequence of interest results in altered gene expression within the host organism. Such alterations in gene expression include, but are not limited to: decreased or enhanced expression of an endogenous gene to manipulate the level of endogenous gene product; newly created expression of a foreign nucleotide sequence to facilitate physiological and/or phenotypic manipulation of an organism; disruption of expression of an endogenous gene to prevent production of the endogenous gene's product; and disrupting expression of an endogenous gene while promoting expression of a nucleotide sequence that encodes a variant of the disrupted endogenous gene product, where expression of the variant gene product confers a desirable attribute to the host organism. Such an endogenous gene may be a gene that is naturally occurring within the organism or a transgene that has previously been integrated into the organism's genome.

Compositions of the invention are novel integration vectors that are derived from cleaved donor complexes (CDCs) of the temperate bacteriophage Mu, a bacterial class III transposon of Escherichia coli. This transposon exhibits extremely high transposition frequency (Toussaint and Resibois (1983) in Mobile Genetic Elements, ed. Shapiro (Academic Press, New York), pp. 105-158). The Mu bacteriophage with its approximately 37 kb genome is relatively large compared to other transposons. Mu encodes two gene products that are involved in the transposition process: MuA transposase, a 70 kDa, 663 amino-acid multidomain protein, and MuB, an accessory protein of approximately 33 kDa. This transposable element has left end and right end recognition sequences (designated “L” and “R,” respectively) that flank the region of the transposon that is ultimately integrated into a site within a target DNA sequence. Unlike other transposons known in the art, these ends are not inverted repeat sequences. The Mu transposable element includes a transpositional enhancer sequence (also referred to herein as the internal activating sequence, or “IAS”), located approximately 950 base pairs inward from the left end recognition sequence.

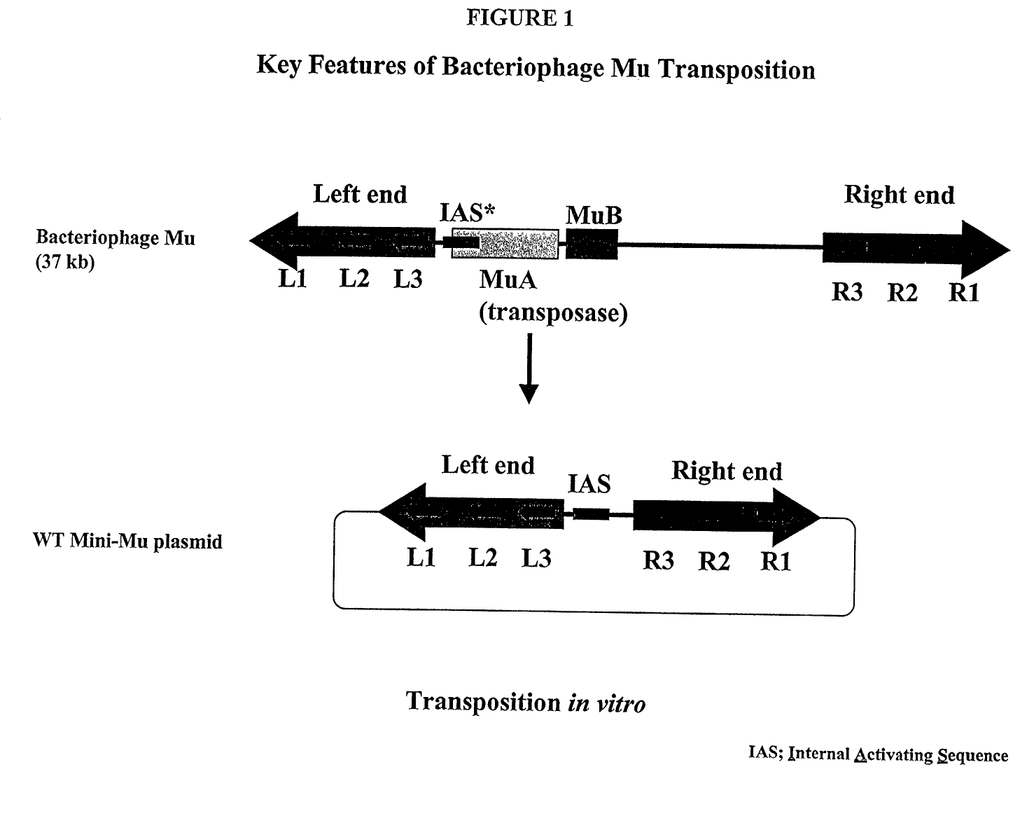

The left and right end recognition sequences of the Mu transposon each encompass three 22-base-pair “end-type”MuA transposase binding sites, attL1 (“L1”), attL2 (“L2”), and attL3(“L3”); and attR1 (“R1”), attR2 (“R2”), and attR3 (“R3”), which are numbered from the extreme ends of the Mu transposable cassette inwards (see FIG. 1). Two dinucleotide DNA cleavage sites reside at the extreme ends of the Mu transposable cassette, that is L1 and R1. The Mu transpositional enhancer sequence also binds the MuA transposase, but at a different domain of the protein than that used to bind the left and right end recognition sequences. MuA transposase interacts with the flanking left and right end recognition sequences and the transpositional enhancer sequence to bring about insertion of the Mu transposable cassette into a target DNA sequence. However, the transpositional enhancer sequence, while part of normal Mu transposition, is not required in the plasmids or integration vectors of the invention. Similarly, other elements which are part of normal Mu transposition may be omitted from the plasmids or integration vectors of the invention so long as the desired result of increased stable transformation is achieved.

Transposition is an essential feature of the Mu life cycle. Integration of infecting Mu DNA into a host chromosome to form a stable lysogen occurs by nonreplicative simple insertion (Liebart et al. (1982) Proc. Natl. Acad. Sci. USA 79:4362-4366; Harshey (1984) Nature 311:580-581). During lytic growth, Mu generates multiple copies of its genome by repeated rounds of replicative transposition (Ljungquist and Bukhari (1977) Proc. Natl. Acad. Sci. USA 74:3143-3147) via a cointegrate pathway (Chaconas et al. (1981) J. Mol. Biol. 150:341-359). Both types of transposition are facilitated by the MuA transposase and accessory MuB protein. E. coli-encoded proteins including histone-like protein (“HU”) and integration host factor (IHF), assist in early conformational changes that ultimately lead to the transfer of the Mu transposable cassette into a target host DNA sequence.

The details of Mu transposition have been elucidated using an in vitro transposition reaction (Mizuuchi (1983) Cell 35:785-794; Mizuuchi (1984) Cell 39:395-404; Craigie and Mizuuchi (1985) Cell 41:867-876; Craigie et al. (1985) Proc. Natl. Acad. Sci. USA 82:750-7574; reviewed by Chaconas et al. (1996) Curr. Biol. 6:817-820; Craigie (1996) Cell 85:137-140; Lavoie and Chaconas (1995) Curr. Topics Microbiol. Immunol. 204:83-99; and Mizuuchi (1992) Annu. Rev. Biochem. 61:1011-1051. In this in vitro reaction, for example, the transposon donor is a mini-Mu plasmid, and another DNA molecule, commonly φX174 replicative form DNA, serves as the target of transposition. The mini-Mu plasmid is constructed such that it comprises two regions of DNA. The first of these regions is a Mu transposable cassette, which is flanked by the second DNA region, referred to herein as the non-Mu plasmid DNA domain (see FIG. 1).

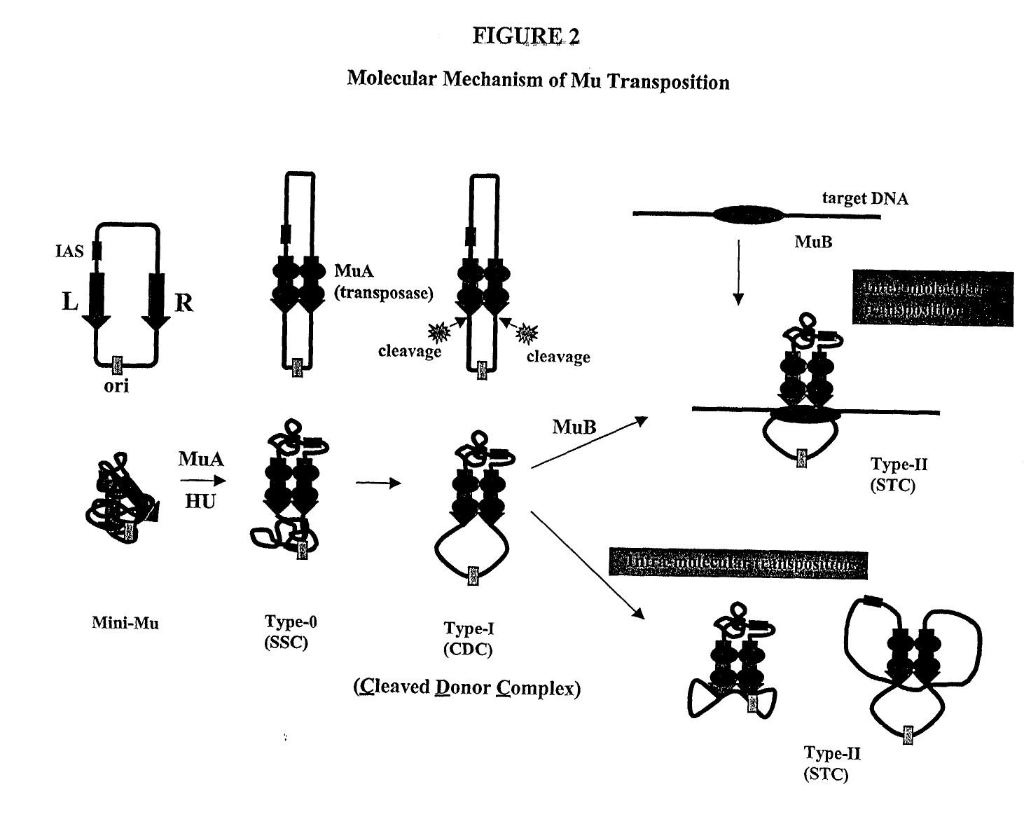

Using this in vitro system, it has been shown that normally MuA transposase exists in its inert monomeric state, which does not recognize the DNA cleavage sites adjacent to the left end and right end recognition sequences of the Mu transposable cassette. Under appropriate conditions, for example, in the presence of HU, IHF, and divalent metal ions, particularly Mg²⁺, MuA transposase initially binds to the Mu transpositional enhancer sequence and to the left and right end recognition sequences. Following this binding, the mini-Mu plasmid undergoes a series of conformational changes that ultimately result in formation of the cleaved donor complex (CDC). In this stable nucleoprotein complex, a single-stranded nick has been introduced at each end of the Mu transposable cassette, exposing 3′ OH groups that act as nucleophiles that attack the target DNA sequence. However, the 5′ ends of the Mu transposable cassette remain attached to the 3′ ends of the non-Mu plasmid DNA (see FIG. 2).

In normal bacteriophage Mu transposition, the structural and functional core of the CDC is a tetrameric unit of MuA molecules (Lavoie et al. (1991) EMBO J. 10:3051-3059; Mizuuchi (1992) Annu. Rev. Biochem. 61:1011-1051; Baker et al. (1993) Cell 74:723-733), hereinafter referred to as the MuA tetrameric core. The three end-type MuA transposase binding sites designated attL1, attR1, and attR2 are considered the core binding sites, as they are stably bound by the MuA tetramer. MuA protein interacting with the other three end-type MuA transposase binding sites (attL2, attL3, and attR3) is loosely bound. These loosely bound MuA molecules can be removed either by heparin, high salt (0.5 M NaCl), or excess Mu end competitor DNA (Kuo et aL (1991) EMBO J. 10:1585-1591; Lavoie et al. (1991) EMBO J. 10:3051-3059; Mizuuchi et al. (1991) Proc. Natl. Acad. Sci. USA 88:9031-9035). These three sites (L1, L2, and L3) are considered accessory sites, as they are dispensable individually and are not required for the intermolecular strand transfer reaction (Allison and Chaconas (1992) J. Biol. Chem. 267:19963-19970; Lavoie et al. (1991) EMBO J. 10:3051-3059; and Mizuuchi et al. (1991) Proc. Natl. Acad. Sci. USA 88:9031-9035. However, sites R1, R2and R3 may be interchanged with sites L1, L2, and L3 for use in constructing plasmids and in preparing the active cleaved donor complexes of the invention.

In the in vitro system in the presence of ATP as well as in bacterial cells, the Mu-encoded protein MuB binds to target DNA in a non-specific manner. Target sites of transposition are determined by MuB. Thus in the in vitro system, MuB binds to the target DNA molecule, while in vivo, it binds to host DNA. The DNA-bound form of MuB has a strong affinity for the Mu CDC, and thus, when present, introduces the CDC to the target molecule or host genome wherever MuB is bound. Because of the non-specific binding of MuB, CDC introduction occurs with little target preference. MuB also stimulates the DNA-breakage and DNA-joining activities of MuA (Adzuma and Mizuuchi (1988) Cell 53:257-266; Baker et al. (1991) Cell 65:1003-1013; Maxwell et al. (1987) Proc. Natl. Acad. Sci. USA 84:699-703; Surette and Chaconas (1991) J. Biol. Chem. 266:17306-17313; Surette et al. (1991) J. Biol. Chem. 266:3118-3124; Wu and Chaconas (1992) J. Biol. Chem. 267:9552-9558; and Wu and Chaconas (1994) J. Biol. Chem. 269:28829-28833). Thus MuB-bound DNA molecules are preferential targets of Mu transposition. In the absence of MuB, introduction of the CDC to a target DNA site still occurs, but is mainly limited to intramolecular reactions which take place in adjacent regions outside the Mu DNA (see FIG. 2).

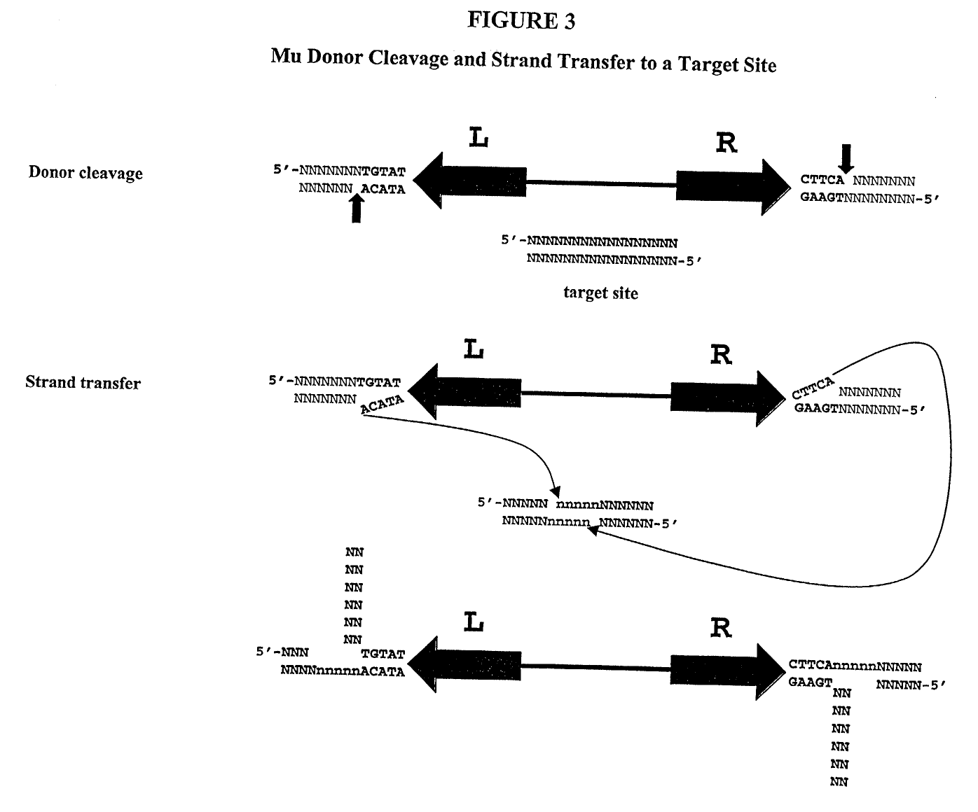

The actual transfer of the Mu transposable cassette from the CDC into a target DNA site is mediated by the bound MuA transposase within the CDC. The exposed 3′ OH ends of the CDC act as nucleophiles, attacking the phosphodiester bond on the backbone of the target DNA. This attacking of a phosphate group by the exposed 3′ OH group forms a bond between the 3′ ends of the Mu DNA and the 5′ ends of the target DNA (see FIG. 3). This process is referred to as strand transfer and results in formation of a strand transfer complex (STC). This stable nucleoprotein complex is involved in both cointegration and simple insertion (see generally, Haren et al. (1999) Ann. Rev. Microbiol. 53: 245-281). Cointegrates are made by replication of the Mu transposable cassette portion of the STC, using the free 3′ ends of the target DNA as primers for leading-strand DNA synthesis. Simple inserts are formed from the STC by degradation of the non-Mu plasmid DNA domain that flanked the Mu transposable cassette portion of the donor molecule, followed by gap repair.

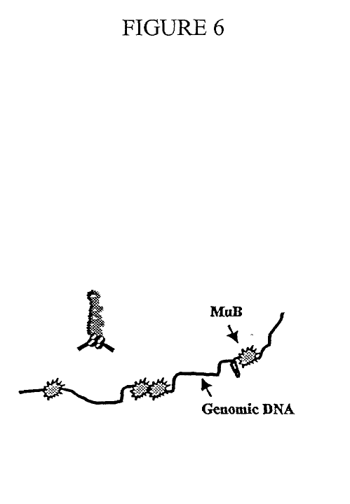

The integration vectors of the present invention comprise Mu bacteriophage “active” cleaved donor complexes. These novel integration vectors allow for stable integration of the entire Mu transposable cassette within the genome of any host organism (depicted in FIGS. 6-8). This integration can occur in the absence of in vivo expression of the MuA transposase protein when the active CDC has the intact MuA tetrameric core attached and so the requirement for such expression is eliminated. This bound MuA transposase is active in strand cleavage and strand transfer in the presence of MuB-bound DNA and hence can provide the molecular machinery for transposition of the Mu transposable cassette into the host DNA. Alternatively, MuA may not always be required to achieve the goals of the invention. By “MuB-bound DNA” is intended sites within the host DNA that are bound by the MuB protein. Such sites become preferred target sites for insertion of the integration vectors of the invention as a result of the high affinity of bound MuB for the MuA tetrameric core associated with the Mu transposable cassette.

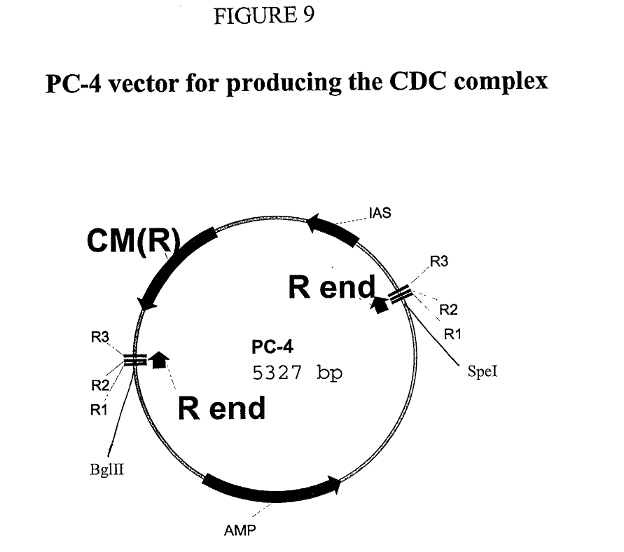

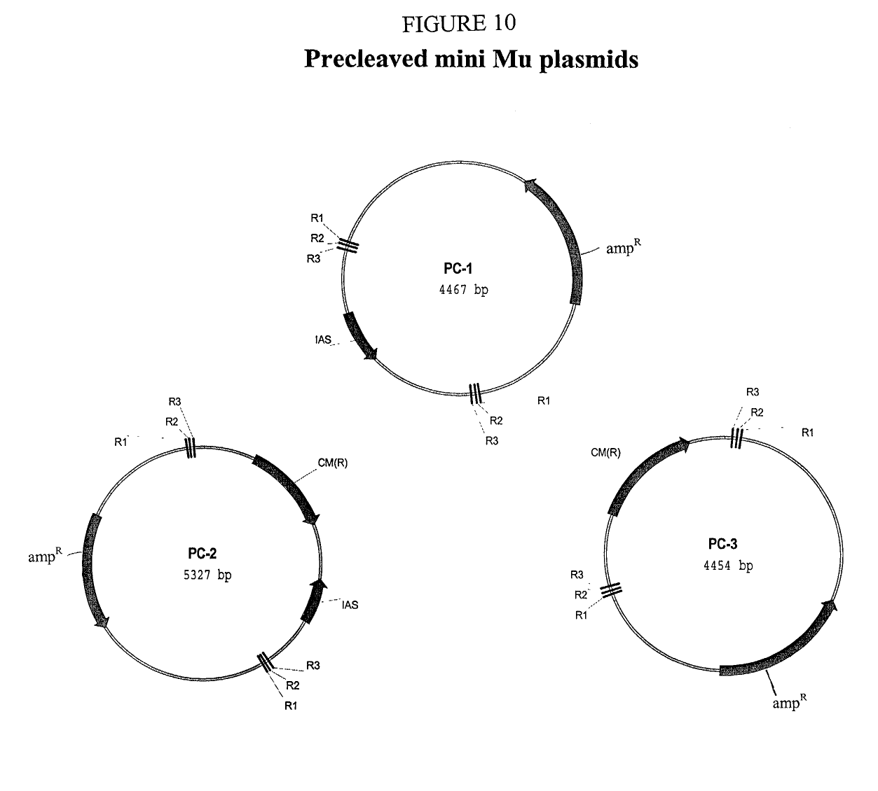

In some embodiments of the invention, cleaved donor complexes (CDCs) are obtained using an in vitro transposition reaction and a mini-Mu plasmid as the transposon donor. By “mini-Mu plasmid” is intended a plasmid comprising a Mu transposable cassette flanked by a non-Mu plasmid DNA domain. See, for example, plasmid PC-4 shown in FIG. 9 and plasmids PC1, PC-2 and PC-3 shown in FIG. 10. Such mini-Mu plasmids can be constructed using molecular biology techniques well known in the art. See particularly Sambrook et al. (1989) Molecular Cloning: A Laboratory Manual (2d ed.; Cold Spring Harbor Laboratory Press, Plainview, N.Y.); and Ausubel et al., eds. (1995) Current Protocols in Molecular Biology (Greene Publishing and Wiley-Interscience, New York).

Any mini-Mu plasmid can be used to obtain the CDCs, so long as the plasmid comprises the necessary DNA elements within the Mu transposable cassette for formation of an active CDC. By “active CDC” is intended a CDC that is capable of carrying out intermolecular strand transfer in an in vitro transposition reaction. Such active CDCs will support intramolecular and/or intermolecular strand transfer in vivo. The necessary elements for active CDC formation depend upon the reaction conditions used during in vitro formation of the CDC (see, for example, Baker and Mizuuchi (1992) Genes and Develop. 6:2221-2232; Wu and Chaconas (1997) J. Mol. Biol. 267:132-141).

Thus, in one embodiment of the invention, an active CDC is obtained using a wild-type mini-Mu plasmid. By “wild-type mini-Mu plasmid” is intended the mini-Mu plasmid has a Mu transposable cassette that comprises the complete Mu left and right end recognition sequences, in their natural (i.e., inverted) orientation; these recognition sequences flank an internal nucleotide sequence comprising the Mu transpositional enhancer sequence. By “complete Mu left and right end recognition sequences” is intended each of the end recognition sequences comprising the three naturally occurring 22-base-pair end-type MuA transposase binding sites. Thus, the left end recognition sequence comprises the attL1, attL2, attL3 end-type MuA transposase binding sites, while the right end recognition sequence comprises the attR1, attR2, and attR3 end-type MuA transposase binding sites. When present, the complete end recognition sequences allow for formation of an active CDC having MuA transposase stably bound to the core binding sites attL1, attR1, and attR2 to form the MuA tetrameric core, and MuA transposase monomers loosely bound to the accessory end-type MuA transposase binding sites attL2, attL3, and attR3. The base pair sequences for the complete Mu left and right end recognition sequences and the Mu transpositional enhancer are known in the art. See Kahmann and Kamp (1979) Nature 280:247-250 and Allet (1978) Nature 274:553-558 for the Mu left end and right end recognition sequences; note, however, that both of these references contain sequencing errors. The correct sequence is found in Genbank Accession No. AF083977 (bacteriophage Mu sequence, contributed by Grimaud (Virology 217: 200-210 (1996) and Morgan et al., direct submission (Aug. 13, 1998)). See also, Mizuuchi and Mizuuchi (1989) Cell 58:399-408 for the Mu transpositional enhancer sequence, herein incorporated by reference. However, one of skill in the art will realize that the exact nucleotide sequence of these recognition sequences may vary slightly, and there is not an exact sequence requirement for functionality of individual binding domains. Thus, for example, the left end recognition sequence comprises three end-type MuA transposase binding sites that reside within nucleotides 1-180 of Genbank Accession No. AF083977, and the right end recognition sequence comprises three end-type MuA transposase binding sites that reside within nucleotides 36641-36662 of Genbank Accession No. AF083977. In one embodiment of the invention, the MuA transposase binding sites in the left end recognition sequence are represented by nucleotides 6-27 (attL1), 111-132 (attL2), and 151-172 (attL3), respectively, of Genbank Accession No. AF083977; and the MuA transposase binding sites in the right end recognition sequence are represented by nucleotides 36691-36712 (attR1), 36669-36690 (attR2), and 36641-36662 (attR3), respectively, of Genbank Accession No. AF083977. One of skill will realize that variations of these sequences may be employed in the invention so long as the desired result is achieved. Thus, sequences having at least about 65%, 70%, 75%, 80%, 85%, 90%, 91%, 92%, 93%, 94%, 95%, 96%, 97%, 98%, 99% or more sequence identity to the native Mu sequences may be employed.

Use of a wild-type mini-Mu plasmid to form an active CDC allows for the in vitro transposition reaction to be carried out under standard reaction conditions. For standard reaction conditions, see Mizuuchi et al. (1992) Cell 70:303-311 and Surette and Chaconas (1992) Cell 68:1101-1108, herein incorporated by reference. When a wild-type mini-Mu plasmid is used in the in vitro transposition reaction under standard conditions, the mini-Mu plasmid must be negatively supercoiled to form an active CDC. However, this requirement for supercoiling under standard reaction conditions can be relieved, for example, by including DMSO in the reaction mixture. See Baker and Mizuuchi (1992) Genes and Develop. 6:2221-2232, herein incorporated by reference.

In another embodiment of the invention, an active CDC is obtained using a derivative mini-Mu plasmid. By “derivative mini-Mu plasmid” is intended a mini-Mu plasmid having a Mu transposable cassette that lacks one or more of the features of the Mu transposable cassette found in a wild-type mini-Mu plasmid. By “features” is intended the following: (1) a complete left end recognition sequence, (2) a complete right end recognition sequence, (3) left and right end recognition sequences in their natural orientation (i.e., inverted), and (4) a Mu transpositional enhancer sequence within the internal nucleotide sequence that is flanked by the left and right end recognition sequences. Thus, for example, a derivative mini-Mu plasmid lacking a complete left or right end recognition sequence lacks one or more of the end-type MuA transposase binding sites within its Mu transposable cassette.

Where a derivative mini-Mu plasmid is used to obtain an active CDC, the reaction conditions required in an in vitro reaction will depend upon what wild-type mini-Mu plasmid feature is missing from the Mu transposable cassette. For example, where the only feature missing is the accessory end-type MuA transposase binding site attR3, standard reaction conditions will yield an active CDC that supports intermolecular strand transfer (Baker and Mizuuchi (1992) Genes and Develop. 6:2221-2232).

Other derivative mini-Mu plasmids having additional features deleted from the Mu transposable cassette can be used to obtain an active CDC by varying the in vitro reaction conditions. For example, when dimethylsulfoxide (DMSO) is included in the transposition reaction under standard reaction conditions, mini-Mu plasmids lacking the Mu transpositional enhancer, carrying only a complete Mu left end or right end recognition sequence, carrying only a single end-type MuA transposase binding site adjacent to a DNA cleavage site with or without the Mu transpositional enhancer, carrying a different (i.e., non-native) combination of MuA transposase binding sites, or having left and right end recognition sequences in direct orientation (rather than inverted orientation) can be used to form a CDC that is active in the DNA cleavage and strand transfer steps required for intermolecular transposition. See Baker and Mizuuchi (1992) Genes and Develop. 6:2221-2232, herein incorporated by reference. A DNA cleavage site may be either a Mu cleavage site or another, possibly artificial cleavage site; for example, a DNA cleavage site may be a restriction enzyme recognition site.

Accordingly, any plasmid or mini-Mu plasmid that yields an active CDC may be used as the basis for obtaining the integration vectors of the invention. Examples of wild-type mini-Mu plasmids that may be used include, but are not limited to, the pBR322-based pBL07 (7.2 kb; Lavoie (1993) in Structural Aspects of the Mu Transpososome (University of Western Ontario, London, Canada); pUC19-based pBL03 (6.5 kb; Lavoie and Chaconas (1993) Genes Dev. 7:2510-2519); pMK586 (Mizuuchi et al. (1991) Proc. Natl. Acad. Sci. USA 88:9031-9035); pMK108 (Mizuuchi (1983) Cell 35:785-794; Craigie and Mizuuchi (1986) Cell 45:793-800); pCL222 (Chaconas et al. (1981) Gene 13:37-46); and pBR322-based pGG215 (7.1 kb; Surette et al. (1987) Cell 49:253-262). Examples of derivative mini-Mu plasmids having one or more MuA binding sites and/or the transpositional enhancer sequence include, but are not limited to, pBL05 (MuA transposase binding site attR3 deleted from pBL03; Allison and Chaconas (1992) J. Biol. Chem. 267:19963-19970); pMK426 (carrying two Mu right end recognition sequences; Craigie and Mizuuchi (1987) Cell 51:493-501); pMK412 (pMK108 with the Mu transpositional enhancer sequence removed; Mizuuchi and Mizuuchi (1989) Cell 58:399-408); and pMK395 (mini-Mu with wrong relative orientation of the two Mu end sequences; Craigie and Mizuuchi (1986) Cell 45:793-800; and others described in Mizuuchi and Mizuuchi (1989) Cell 58:399-408, herein incorporated by reference. Also suitable for formation of an active mutant CDC are pUC19 derivatives carrying specific MuA-binding sites, such as the derivatives described by Baker and Mizuuchi et al. (1992) Genes and Develop. 6:2221-2232. All of the foregoing references describing such mini-Mu plasmids are herein incorporated by reference.

To obtain the integration vectors of the invention, a wild-type mini-Mu plasmid or a derivative mini-Mu plasmid is constructed that comprises a nucleotide sequence of interest within a Mu transposable cassette, and optionally a pair of restriction sites at each end of the non-Mu plasmid DNA domain, adjacent to the extreme ends of the Mu transposable cassette. When present, these restriction sites may allow for removal of the non-Mu plasmid DNA domain following formation of the active CDC. By “nucleotide sequence of interest” is intended any nucleotide sequence that, when delivered into a host cell using the integration vector of the invention, results in a desired change within the host organism. Such nucleotide sequences of interest are defined in more detail below. Where in vitro production of active CDC is desired, the mini-Mu plasmid may then be subjected to an in vitro reaction to form an active cleaved donor complex (CDC).

Methods for producing active CDCs are well known in the art. See particularly Craigie et al. (1985) Proc. Natl. Acad. Sci. USA 82:7570-7574; Wu and Chaconas (1997) J. Mol. Biol. 267:132-141, herein incorporated by reference. This in vitro reaction may be carried out under standard reaction conditions (Craigie et al. (1985) Proc. Natl. Acad. Sci. USA 82:7570-7574, herein incorporated by reference) or under modified reaction conditions (such as with the addition of DMSO or glycerol; see, for example, Mizuuchi and Mizuuchi (1989) Cell 58:399-408, herein incorporated by reference) to obtain an active CDC. Active CDCs may be obtained in vivo (i.e., in the host cell) where MuA is introduced into or expressed in a cell in which DNA from a mini-Mu plasmid or other plasmid capable of forming an active CDC is also present. In some embodiments, for example, formation of active CDCs from DNA of a mini-Mu plasmid previously integrated into the genome of the host organism could result in deletion of most of the previously integrated DNA and could also result in integration of the active CDC into a new location in the host genome.

For example, where in vitro production of active CDCs is desired, a mini-Mu plasmid of interest is incubated with the purified native MuA transposase protein and the E. coli HU protein, or biologically active variants or fragments thereof as defined below, in the presence of a divalent metal ion such as Mg²⁺ or Mn²⁺ (Mizuuchi et al. 1992 Cell 70:303-311). Where the Mu transposable cassette comprises a Mu transpositional enhancer sequence, the purified E. coli protein IHF or variant thereof is also included in the incubation reaction. Following formation of the CDC, the reaction is terminated by addition of EDTA (see Wu and Chaconas (1997), J. Mol. Biol. 267:132-141) to obtain the stable active CDC. Further spontaneous rearrangements of the CDC can also be inhibited by incubation at 0° C. (see Surette et al. (1987) Cell 49:253-262). Where the CDC has been derived from a wild-type mini-Mu plasmid, the loosely bound MuA transposase molecules may be removed to obtain a stripped down version of the active CDC (Wu and Chaconas (1997) J. Mol. Biol. 267:132-141). This stripped-down active CDC may be used for preparing the integration vectors of the invention. Removal of this loosely bound MuA makes intramolecular transposition a less efficient process, which would be advantageous for methods of the invention directed to intermolecular events. However, when the active CDC is intact, and thus comprises the MuA tetrameric core and MuA transposase molecules loosely bound to the accessory binding sites attL2, attL3, and attR3, intermolecular strand transfer occurs four times faster than with the stripped-down CDC (Wu and Chaconas (1997) supra). Thus, when a stripped-down CDC is to be used, additional MuA protein can be codelivered into the host cell to promoter intermolecular strand transfer. Additional MuA can be codelivered directly using a technique such as microinjection or particle bombardment or it may be codelivered indirectly by delivering an expression vector comprising the MuA coding sequence operably linked to regulatory elements that promote expression in the host cell. Since MuA must be imported into the nucleus, such a DNA construct would further comprise a sequence encoding a nuclear localization signal, such as the SV40 NLS, fused in frame with the MuA coding sequence. In addition to MuA, other proteins or compounds may be helpful in achieving the desired results of stable integration of the CDC, and such proteins or compounds may also be codelivered into the host cell with the vectors of the present invention.

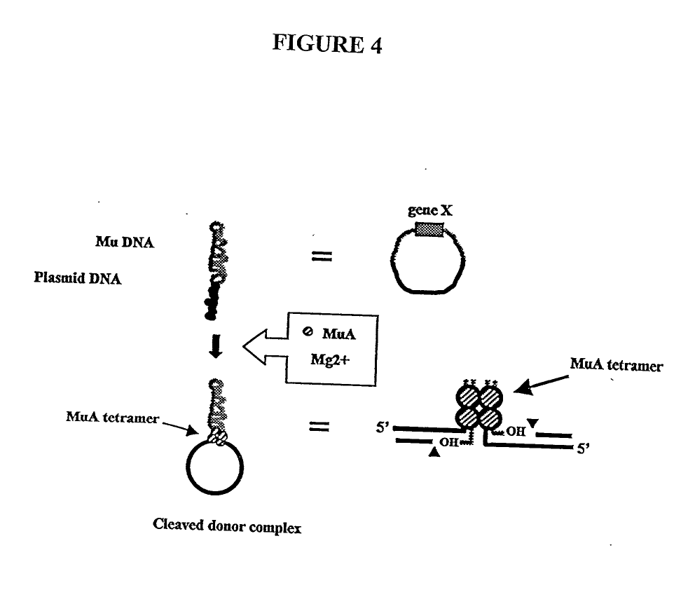

Thus, a mini-Mu plasmid of interest and the native MuA transposase, HU, and IHF proteins, or biologically active variants or fragments thereof, may be used in an in vitro reaction under standard or modified reaction conditions to obtain a stable active CDC that is active in intermolecular transposition (see FIG. 4). During formation of this CDC, a nick has been introduced at each end of the Mu transposable cassette, exposing 3′-OH groups, relaxing the non-Mu plasmid domain of the mini-Mu plasmid (see FIGS. 2 and 3). This stable CDC may then serve as an integration vector of the invention, or may then be modified within the non-Mu plasmid DNA domain prior to use as an integration vector.

Thus, in some embodiments, the resulting CDC is digested with a restriction enzyme that recognizes restriction enzyme sites within the non-Mu plasmid DNA domain adjacent to the extreme ends of the Mu transposable cassette. Double-stranded cleavage at these two sites removes the non-Mu plasmid DNA domain, leaving the Mu transposable cassette having MuA bound to form the MuA tetrameric core and also two exposed 3′-OH ends.

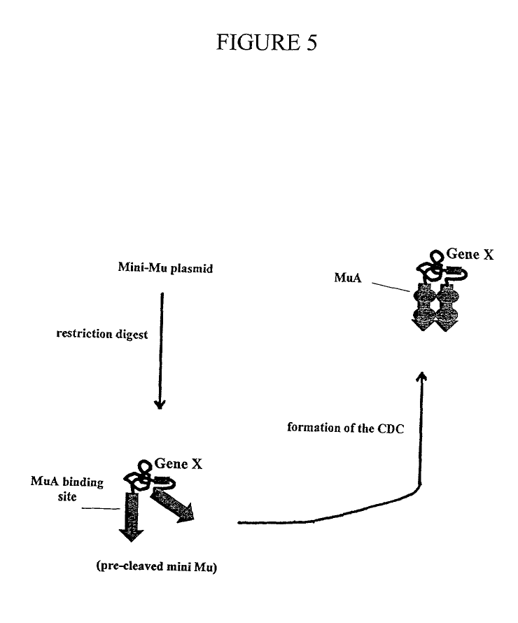

In some embodiments of the present invention, the novel integration vectors are CDCs that are obtained from precleaved or “precut” mini-Mu plasmids. By “precleaved” or “precut” mini-Mu plasmid is intended a wild-type or derivative mini-Mu plasmid that has been subjected to restriction enzyme digestion to cleave the double-stranded non-Mu plasmid DNA domain within at least one region, thereby linearizing this domain prior to formation of the active CDC. In some embodiments a derivative mini-Mu plasmid is used, for example, a derivative mini-Mu plasmid that comprises two Mu right end recognition sequences in their natural inverted orientation. Strand transfer is most efficient when a pair of Mu right end recognition sequences is used with precleaved miniMu plasmids. See, for example, Craigie and Mizuuchi (1987) Cell 51:493-501, and Namgoong et al (1994) J. Mol. Biol. 238:514-527. Each of these Mu right end recognition sequences can be the complete Mu right end recognition sequence, i.e., having all three end-type MuA transposase binding sites (i.e., attR1, attR2, and attR3) in natural orientation and order, or can comprise just one or more sites, for example, the attR1 and attR2 sites in their natural orientation (see Savilahti et al. (1995) EMBO J. 14:4893-4903). As with other mini-Mu plasmids used in the transformation methods of the invention, the Mu end recognition sequences flank an internal nucleotide sequence, which, for purposes of the present invention, has been engineered to comprise a nucleotide sequence of interest to be stably integrated within the genome of a host organism. The nucleotide sequence of interest may be, for example, a gene of interest, including a scorable marker gene, or other sequence of interest described herein below. The internal nucleotide sequence may further comprise, for example, the Mu transpositional enhancer sequence. In some embodiments, the restriction enzyme is chosen such that double-stranded cleavage takes place within a region of nucleotides adjacent to the extreme end of the Mu transposable cassette. Where restriction sites have been engineered at each end of the non-Mu plasmid DNA domain, adjacent to the extreme ends of the Mu transposable cassette, removal of the entire non-Mu plasmid DNA domain can take place prior to formation of an active CDC (see, for example, FIG. 5). For preparation and use of precleaved mini-Mu plasmids (also referred to as precleaved Mu DNA), see Craigie and Mizuuchi (1987) Cell 51:493-501; Mizuuchi and Mizuuchi (1989) Cell 58:399-408; Savilahti et al. (1995) EMBO J. 14:4893-4903; Haapa et al. (1999) Nucleic Acids Res. 27:2777-2784; and Haapa et al. (1999) Genome Res. 9:308-315.

The precleaved mini-Mu plasmid may then be subjected to an in vitro transposition reaction as previously described to obtain an active CDC. This embodiment of the invention is similar in concept to the active CDCs obtained from wild-type or derivative mini-Mu plasmids described elsewhere herein, but offers potential advantages because the in vitro transposition reaction requirements for formation of an active CDC from a precleaved mini-Mu plasmid are relaxed (see, for example, Craigie and Mizuuchi (1987) Cell 51:493-501, and Mizuuchi and Mizuuchi (1989) Cell 58:399-408). Thus, active CDC formation can take place in the absence of the E. coli HU protein and superhelicity of the mini-Mu plasmid. The elimination of these requirements is beneficial because HU is not readily commercially available and isolation of plasmids with high superhelicity is more time consuming and labor intensive. Further, where removal of the non-Mu plasmid DNA domain is desired, some embodiments make use of the precleaved mini-Mu plasmid so that restriction digestion following CDC formation can be avoided, as such removal occurs prior to formation of the CDC. Thus, use of the precleaved mini-Mu plasmids can minimize manipulation, and potentially prolonged incubation, of the CDC in different environments.

Thus, the novel integration vectors of the invention may be obtained using mini-Mu plasmids as well as any other necessary or helpful proteins, such as, for example, MuA transposase and the bacterial proteins HU and IHF, or biologically active variants or fragments thereof. Such proteins may be produced in the host genome, for example as the result of previous genetic engineering of the genome, or the proteins may be introduced along with the integration vectors during or after transformation of the host genome with the integration vectors. Such introduction may be direct or indirect (for example, by cotransformation of an integration vector with another DNA sequence encoding MuA transposase). Thus, active CDCs may be formed within the host cell where the appropriate elements and sequences exist within the transformed host cell or cell derived from a transformed host cell. Where purified proteins are to be used, methods for obtaining these purified native proteins or biologically active variants or fragments thereof are known in the art. See, for example, Craigie and Mizuuchi (1985) J. Biol. Chem. 260:1832-1835 (cloning of the MuA gene and purification of MuA); Craigie et al. (1985) Proc. Natl. Acad. Sci. USA 82:7570-7574; Rouviere-Yaniv and Gros (1975) Proc. Natl. Acad. Sci. USA 72:3428-3432, Dixon and Kornberg (1984) Proc. Natl. Acad. Sci. USA 81:424-428, and Surette et al. (1987) Cell 49:253-262 (purification of HU); Wu and Chaconas (1994) J. Biol. Chem. 269:28829-28833, and the references cited therein (MuA, HU, and IHF); Yang et al. (1995) EMBO J. 14:2374-2384 (native MuA and variants thereof, and HU); herein incorporated by reference.

By “purified” is intended the protein, or biologically active variant or fragment thereof, is substantially or essentially free from components that normally accompany or interact with the protein as found in its naturally occurring environment. Thus a purified protein is substantially free of other cellular material, or culture medium when produced by recombinant techniques,.or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the MuA, HU, or IHF, or biologically active variant or fragment thereof is recombinantly produced, in some embodiments, culture medium represents less than about 30%, 20%, 10%, 5%, or 1% (by dry weight) of chemical precursors or non-protein-of-interest chemicals.

By “fragment” is intended a portion of the amino acid sequence and hence protein encoded thereby. A biologically active portion of the MuA, HU, or IHF protein can be prepared by isolating a portion of their respective coding sequences, expressing the encoded portion of the respective protein (e.g., by recombinant expression in vitro), and assessing the activity of the encoded portion of the respective protein. The coding sequences for these proteins are known in the art. See, for example, Grimaud (1996) Virology 217(1):200-210 for the nucleotide sequence for the Mu bacteriophage (GenBank Accession No. AF083977), which identifies the coding sequence for the MuA transposase (GenBank Accession No. AAF01083) and MuB protein (GenBank Accession No. AAF01100); Miller (1984) Cold Spring Harb. Symp. Quant. Biol. 49:691-698 for the coding sequence for the IHF alpha-subunit (GenBank Accession No. P06984) and Flamm and Weisberg (1985) J. Mol. Biol. 183(2):117-128 for the coding sequence for the IHF beta-subunit (GenBank Accession No. P08756); and GenBank Accession No. U82664, nucleotides 40901-41173, which code for the HU protein (GenBank Accession No. AAB40196).

By “variant” MuA, HU, or IHF protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant MuA transposase, HU, and IHF proteins useful in carrying out the construction of the integration vectors of the invention are biologically active, that is they continue to possess the desired biological activity of the native protein. Thus, a variant MuA transposase effectively binds to the attachment sites of the native or mutant CDC and assists with intermolecular transposition of the Mu transposable cassette from the integration vector of the invention into a predetermined target site within the genome of a host organism. Similarly, a variant HU protein interacts with a mini-Mu plasmid donor near an attL1 attachment site within the Mu transposable cassette to facilitate the role of this attachment site in CDC assembly. A variant IHF protein, when present in the reaction mixture, binds to its specific site in the Mu transpositional enhancer sequence to achieve the optimal geometrical conformation of the mini-Mu plasmid domain comprising the Mu transposable cassette. Such variant proteins may result from, for example, genetic polymorphism or from human manipulation.

The known amino acid sequences for the native MuA transposase, HU, IHF, and other proteins may be altered in various ways including amino acid substitutions, deletions, truncations, and insertions. Methods for such manipulations are generally known in the art. For example, amino acid sequence variants of these proteins can be prepared by mutations in their respective coding sequences. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and references cited therein. Guidance as to appropriate amino acid substitutions that do not affect biological activity of the protein of interest may be found in the model of Dayhoff et al. (1978) Atlas of protein Sequence and Structure (Natl. Biomed. Res. Found., Washington, D.C.), herein incorporated by reference. Conservative substitutions, such as exchanging one amino acid with another having similar properties, may be preferred.

Thus, the MuA transposase, HU, IHF, and other proteins used to obtain the integration vectors of the invention include both the native (i.e., naturally occurring) proteins as well as biologically active variants. Obviously, where mutations are made in their respective DNA coding sequences to obtain variant forms, the mutations must not place the sequence out of reading frame and preferably will not create complementary regions that could produce secondary mRNA structure. See, EP Patent Application Publication No. 75,444.

The deletions, insertions, and substitutions of the amino acid sequences for these proteins are not expected to produce radical changes in the characteristics of the respective proteins. However, when it is difficult to predict the exact effect of the substitution, deletion, or insertion in advance of doing so, one skilled in the art will appreciate that the effect will be evaluated by routine screening assays. Thus, activity of variant MuA transposase, HU, and IHF proteins can be evaluated using the standard in vitro transposition reaction (Mizuuchi et al. (1992) Cell 70:303-311); herein incorporated by reference.

Where standard in vitro reaction conditions are to be used for producing active CDCs, variant MuA transposase proteins may retain amino acid residues 1-76 of the native MuA protein (the so-called N-terminal domain). However, when DMSO is included in the standard reaction conditions, active CDCs can be obtained using variant MuA transposase proteins lacking the N-terminal domain. See Mizuuchi and Mizuuchi (1989) Cell 58:399-408.

Biologically active variants of a native MuA transposase, HU, IHF, or other protein will have at least about 40%, 50%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity to the amino acid sequence for the native protein as determined by sequence alignment programs described below using default parameters. A biologically active variant of these proteins may differ from the native protein by as few as 1-15 amino acid residues, as few as 1-10, such as 6-10, as few as 5, as few as 4, 3, 2, or even 1 amino acid residue.

The following terms are used to describe the sequence relationships between two or more polypeptides: (a) “reference sequence”, (b) “comparison window”, (c) “sequence identity”, (d) “percentage of sequence identity”, and (e) “substantial identity”.

(a) As used herein, “reference sequence”is a defined sequence used as a basis for sequence comparison. A reference sequence may be a subset or the entirety of a specified sequence; for example, a segment of a full-length amino acid sequence, or the complete amino acid sequence.

(b) As used herein, “comparison window” makes reference to a contiguous and specified segment of an amino acid sequence, wherein the amino acid sequence in the comparison window may comprise additions or deletions (i.e., gaps) compared to the reference sequence (which does not comprise additions or deletions) for optimal alignment of the two sequences. Generally, the comparison window is at least 20 contiguous amino acids in length, and optionally can be 30, 40, 50, 100, or longer. Those of skill in the art understand that to avoid a high similarity to a reference sequence due to inclusion of gaps in the amino acid sequence a gap penalty is typically introduced and is subtracted from the number of matches.

Methods of alignment of nucleotide and amino acid sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a MuA transposase, HU, or IHF protein of the invention. BLAST protein searches can be performed with the BLASTX program, score=50, wordlength=3, to obtain amino acid sequences homologous to these proteins. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

Unless otherwise stated, sequence identity/similarity values provided herein refer to the value obtained using GAP version 10 using the following parameters: % identity using GAP Weight of 50 and Length Weight of 3; % similarity using Gap Weight of 12 and Length Weight of 4, or any equivalent program. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48: 443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the amino acid sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity.

(e) The term “substantial identity” in the context of a peptide indicates that a peptide comprises an amino acid sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman and Wunsch (1970) J. Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

The novel integration vectors of the invention are useful in methods of the invention directed to genetic manipulation of a host organism's genome. In this manner, a host cell is transformed with an integration vector of the invention, which comprises a nucleotide sequence of interest, and with a plasmid comprising a coding sequence for the MuB accessory protein or biologically active variant or fragment thereof. The coding sequence for the MuB accessory protein is operably linked to a promoter that drives expression in the host cell.

Thus, the integration vectors of the invention are derived from, for example, a mini-Mu plasmid or precleaved mini-Mu plasmid comprising at least one nucleotide sequence of interest within the Mu transposable cassette. By “nucleotide sequence of interest” is intended a sequence that codes for a desired RNA or protein product, or which itself provides the host cell with a desired property, i.e., a mutant phenotype. Thus, for example, a nucleotide sequence of interest may be a sequence encoding a structural or regulatory protein, or may be a regulatory sequence such as a promoter. The desired RNA or protein product or regulatory sequence may be heterologous, i.e., foreign, or it may be native to the host cell. If the sequence is native to the host cell, transformation of the host cell with the sequence results in a change in phenotype. Where appropriate, the nucleotide sequence of interest may also comprise one or more regulatory elements required for or involved in the expression of the nucleotide sequence encoding the desired RNA or protein product, such as a promoter, a terminator, and the like. The regulatory element(s) may be either heterologous or homologous to the DNA sequence of interest. In this manner, the nucleotide sequence of interest may be constructed as part of an expression cassette as described elsewhere herein and inserted within the internal DNA sequence of the Mu transposable cassette.

Thus, for example, where the host cell is a bacterial or yeast cell, the integration vector of the invention may comprise a nucleotide sequence coding for a polypeptide of interest. Transformation of the bacterial or yeast host cell with such an integration vector allows for stable insertion of the coding sequence and its regulatory regions within the host genome. Following their selection, transformed host cells are cultivated in a suitable nutrient medium under conditions permitting the expression of the polypeptide, after which the resulting polypeptide is recovered from the culture.

Media used to culture the cells may be any conventional media suitable for growing the cells, such as minimal or complex media containing appropriate supplements. Suitable media are available from commercial suppliers or may be prepared according to published recipes (e.g., in catalogues of the American Type Culture Collection). The polypeptide produced by the cells may then be recovered from the culture medium by conventional procedures including separating the cells from the medium by centrifugation or filtration, precipitating the proteinaceous components of the supernatant or filtrate by means of a salt, e.g. ammonium sulfate, purification by a variety of chromatographic procedures, e.g. ion exchange chromatography, gel filtration chromatography, affinity chromatography, or the like, depending on the type of polypeptide in question.

Where the host cell is a bacterial or yeast cell to be utilized in production of a polypeptide of interest, the polypeptide may be a translocated polypeptide. By “translocated polypeptides” is intended the polypeptide, when expressed, carries a signal seuqence which enables it to be translocated across the cell membrane, thereby facilitating its recovery from the culture medium.

Of particular interest are plants that have been transformed with an integration vector of the invention to achieve stable integration of a nucleotide sequence of interest within the plant's genome. For this intended purpose, the nucleotide sequence of interest may be a regulatory sequence, such as a desirable promoter sequence; recombination sites that allow for subsequent targeted insertion of another nucleotide sequence of interest using, for example, the FLP/FRT or Cre/lox recombination systems discussed elsewhere herein; an antisense sequence or sense sequence for a gene of interest that allows for suppression of expression of the gene product; or a gene encoding a protein whose expression confers a desirable phenotype in the transformed plant. Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. application Ser. No. 08/838,763, filed Apr. 10, 1997; and U.S. Pat. Nos. 5,703,049, 5,885,801, and 5,885,802, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworrn, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); lectins (Van Damme et al. (1994) Plant Mol. Biol. 24:825); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes encoding resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes encoding resistance to herbicides that act to inhibit action of glutamine synthase, such as phosphinothricin or basta (e.g., the bar gene), or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in copending U.S. application Ser. No. 08/838,763, filed Apr. 10, 1997, and U.S. Pat. Nos. 5,703,049, 5,885,801, and 5,885,802.

Commercial traits may also be encoded by a gene or genes that could increase, for example, starch for ethanol production, or provide expression of proteins. Another important commercial use of transformed plants is the production of polymers and bioplastics such as described in U.S. Pat. No. 5,602,321. Genes such as β-ketothiolase, PHBase (polyhydroxybutyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

The nucleotide sequence of interest may be an antisense sequence or sense sequence for an endogenous gene thereby providing a method for suppression of expression of an endogenous gene. Methods for suppressing gene expression using antisense sequences are known in the art. For example, antisense constructions, complementary to at least a portion of the messenger RNA (MRNA) for the targeted endogenous gene sequence can be constructed. Antisense nucleotides are constructed to hybridize with the corresponding mRNA. Modifications of the antisense sequences may be made as long as the sequences hybridize to and interfere with expression of the corresponding MRNA. In this manner, antisense constructions having 70%, 80%, 85%, 90%, 95%, and higher sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

Methods for sense suppression generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, preferably greater than about 65% sequence identity or about 85% sequence identity, or greater than about 95% or greater sequence identity. See, U.S. Pat. Nos. 5,283,184 and 5,034,323; herein incorporated by reference.

The mini-Mu plasmid serving as the starting point for formation of an integration vector of the invention may be constructed such that the internal DNA sequence of the Mu transposable cassette further comprises a scorable marker gene to facilitate selection of transformed host cells comprising the Mu transposable cassette inserted within the predetermined target site. See, for example, Chaconas et al. (1981) Gene 13:3746. Scorable marker genes include, for example, selectable marker genes and assayable reporter genes.

Selectable marker genes confer resistance to a particular selection agent, and thus allow for selection of transformed cells/tissues in the presence of such a selection agent. Selectable marker genes include, but are not limited to, genes encoding antibiotic resistance, such as those encoding neomycin phosphotransferase II (NEO) (Fraley et al. (1986) CRC Critical Review in Plant Science 4:1-25) and hygromycin phosphotransferase (HPT or HYG) (Vanden Elzen et al. (1985) Plant Mol. Biol. 5:299; Shimizu et al. (1986) Mol. Cell Biol. 6:1074, as well as genes conferring resistance to herbicidal compounds, such as glufosinate ammonium, bromoxynil, and 2,4-dichlorophenoxyacetate (2,4-D).

By “assayable reporter gene” is intended any scorable marker gene, other than a selectable marker gene, that can be assayed for its presence and/or expression. Reporter genes generally encode a protein whose activity can be assayed to determine whether the reporter gene is present and/or is being expressed. In some embodiments, the protein is assayed using nonlethal methods. Use of such assayable reporter genes as opposed to selectable marker genes to facilitate selection of transgenic plants is disclosed in detail in the copending application entitled “Recovery of Transformed Plants Without Selectable Markers by Nodal Culture and Enrichment of Transgenic Sectors,” U.S. patent application Ser. No. 08/857,664, filed May 16, 1997, herein incorporated by reference.

With assayable reporter genes, generally too there is some sort of chemical, biological, or physical assay available that will determine the presence or absence or change in amount of the expression product of the gene. In certain embodiments in which the assayable reporter gene produces an enzyme involved in a metabolic pathway, the assay may determine the presence or absence of, or a change in the amount of, a metabolite produced directly by the enzyme, or the presence or absence of, or a change in the amount of, a metabolite produced indirectly by the enzyme, or the presence or absence of, or a change in the amount of, the final product of the metabolic pathway, rather than the presence or absence of the expression product (the enzyme) itself. For example, such an enzyme might be involved in a metabolic pathway that produces oils having a particular fatty acid makeup. It will also be apparent to those of skill in the art that many forms of assay techniques are available to detect the presence and/or expression of reporter genes. For example, any expressed protein capable of detection by ELISA could be assayed by using the associated ELISA, or a modification in the amount of a specific fatty acid could be determined using the appropriate biochemical analytical technology (GCMS, for example); or a bioassay could be used (for example, expression of a crystal protein toxin from Bacillus thuringiensis (Bt) could be determined by screening for deleterious effects of transformed plant tissue on insects or insect larvae that are susceptible to the crystal protein toxin).

Those of skill in the art will also recognize that the presence of the assayable reporter gene can be detected directly using DNA amplification techniques known in the art, including, but not limited to PCR, RT-PCR, or LCR, for example. By way of illustration, the assayable reporter gene could be an embryo-specific gene such as a desaturase under the control of an embryo-specific promoter. Genetic modification using such a gene construct would be expected to modify seed oil profiles, without affecting expression in leaves. A properly performed PCR screen would detect the presence of the sequence in transformed plants. It will also be recognized that any gene that can be amplified using amplification technology such as PCR can serve as an assayable reporter gene in the present invention.

Reporter genes are particularly useful to quantify or visualize the spatial pattern of expression of a gene in specific tissues. Commonly used reporter genes include, but are not limited to, β-glucuronidase (GUS) (Jefferson (1987) Plant Mol. Biol. Rep. 5:387); B-galactosidase (Teeri et al. (1989) EMBO J. 8:343-350); luciferase (Riggs et al. (1987) Nucleic Acids Res. 15(19): 8115; Luehrsen et al. (1992) Methods Enzymol. 216: 397-414); chloramphenicol acetyltransferase (CAT) (Lindsey and Jones (1987) Plant Mol. Biol. 10:43-52); green fluorescence protein (GFP) (Chalfie et al. (1994) Science 263:802); and the maize genes encoding for anthocyanin production (Ludwig et al. (1990) Science 247:449).

Other examples of assayable reporter genes include, but are not limited to, the oxalate oxidase gene, which has been isolated from wheat (Dratewka-Kos et al. (1989) J. Biol. Chem. 264:4896-4900 (the “germin” gene) and barley (WO 92/14824); the oxalate decarboxylase gene, which has been isolated from Aspergillus and Collybia (see WO 94/12622); other enzymes that utilize oxalate; other enzymes such as polyphenol oxidase, glucose oxidase, monoamine oxidase, choline oxidase, galactose oxidase, 1-aspartate oxidase, and xanthine oxidase, and the like.

As those of skill in the art will recognize, the assay for reporter genes will vary with the nature of the expression product. For example, an enzymatic assay can be used in those instances where the expression product is an enzyme, such as in the case of transformation with a gene encoding oxalate oxidase or oxalate decarboxylase. A visual or calorimetric assay would be appropriate for cells or tissues transformed with a GFP gene. As those skilled in the art will also recognize, when an enzymatic assay is appropriate, the existence of an assay in the art would be particularly useful. Furthermore, as noted above, other assay techniques (e.g., PCR for the assayable reporter itself, or ELISA, or a bioassay, or chemical analytical methods such as GCMS) will be appropriate in the performance of the various embodiments of the invention.

In a further alternative embodiment of the present invention, the assay can involve a procedure that measures a loss of, or a decrease in the level of expression of, a measurable product that is normally present or that is normally expressed at higher levels. For example, gene disruption may decrease or eliminate gene expression from a particular gene copy, and antisense or co-suppression technology can be used to downregulate the expression of a particular gene. An appropriate assay that would detect the disappearance of or decease in amount of the expression product or a metabolic product can be used.

For further information on the use of scorable marker genes, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; M. Gossen (1993) Ph.D. Thesis, University of Heidelberg; Reines et al. (1993) Proc. Natl. Acad. Sci. USA 90:1917-1921; Labow et al. (1990) Mol. Cell Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res, 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Gatz et al. (1992) Plant J. 2:397-404; A. L. Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook Exp. Pharmacol. 78; Gill et al. (1988) Nature 334:721-724. Such disclosures are herein incorporated by reference.

When present in the Mu transposable cassette, the scorable marker gene is operably linked to regulatory regions, i.e., to a promoter and terminator sequence, that drive expression of the scorable marker within an organism transformed with the novel integration vectors of the invention. Thus the scorable marker gene can be constructed as part of an expression cassette as described elsewhere herein and inserted within the internal DNA sequence of the Mu transposable cassette.

Subsequent removal of the scorable marker gene from stably transformed cells may be of interest, such as when the marker gene is undesirable, e.g., from an environmental point of view. Thus, in one embodiment of the invention, the scorable marker gene within the Mu transposable cassette is engineered with flanking target sequences for a site-specific recombination enzyme. These flanking target sequences may or may not be identical so long as the recombinase protein is capable of recognizing and interacting with the target sequences. The presence of these target sequences allows for the specific deletion or knockout of the marker gene from the genome of a host cell having the Mu transposable cassette integrated within its genome. This enables construction of marker-free transformed cells having the desired phenotypic change. In this embodiment of the invention, the desired outcome from transformation with an integration vector of the invention is achieved in a two-step process. In the first step, integration of the Mu transposable cassette into the genome of the host cell is accomplished by transformation and selection of host cells testing positive for the scorable marker gene. In the second step, removal of the marker gene from the host genome is accomplished by a site-specific recombinase, which interacts with the target sequences flanking the marker gene.

The site-specific recombinase can be provided in cis with the scorable marker sequence, i.e., within the Mu transposable cassette comprising the scorable marker, or in trans, i.e., on a different transformation vector. When provided in cis, the recombinase DNA sequence should be operably linked to an inducible promoter, such as for example, chemical-inducible promoters and temperature-inducible promoters. In this manner, expression of the recombinase protein can be controlled to take place only after targeted integration has taken place. Where the DNA sequence encoding the recombinase enzyme is provided in cis and under the control of an inducible promoter, targeted integration of the Mu transposable cassette and subsequent removal of the scorable marker gene may be accomplished using only one transformation step.

Several site-specific recombination systems are known in the art, all of which are encompassed for the intended use of removing an undesirable scorable marker gene following targeted integration of the Mu transposable cassette within the host organism's genome. In some embodiments of the present invention, the site-specific recombination system consists of a site-specific recombinase enzyme and a target sequence for said enzyme. The recombination system of choice will depend upon the host organism. Examples of such systems are the pAM O1 resolvase having as target sequence the pAMβ1 res sequence (Janniere et al. (1993) in Bacillus subtilis and Other Gram-positive Bacteria: Biochemistry, Physiology and Molecular Genetics, ed. Sonenshein et al. (American Society for Microbiology, Washington, D.C.), pp. 625-644); the phage P1 Cre enzyme having as target sequence the P1 lox site (Hasan et al. (1994) Gene 150:51-56); and the yeast FLP recombinase enzyme having as target sequence the FRT site (Cox (1993) Proc. Natl. Acad. Sci. USA 80:4223-4227). When the host organism is a plant, the recombinase system may be the FLP/FRT or Cre/lox system.

The FLP recombinase is a protein that catalyzes a site-specific reaction that is involved in amplifying the copy number of the 2μ plasmid of Saccharomyces cerevisiae during DNA replication. FLP protein has been cloned and expressed. See, for example, Cox (1993) Proc. Natl. Acad. Sci. USA 80:4223-4227, herein incorporated by reference. The FLP recombinase for use in the invention may be that derived from the genus Saccharomyces. It may be preferable to synthesize the recombinase using plant-preferred codons for optimum expression in a plant of interest. See U.S. Pat. No. 5,929,301, herein incorporated by reference. The bacteriophage recombinase Cre catalyzes site-specific recombination between two lox sites. The Cre recombinase is known in the art. See, for example, Guo et al. (1997) Nature 389:40-46; Abremski et al. (1984) J. Biol. Chem. 259:1509-1514; Chen et al. (1996) Somat. Cell Mol. Genet. 22:477-488; and Shaikh et al. (1977) J. Biol. Chem. 272:5695-5702; herein incorporated by reference. The Cre recombinase may also be synthesized using plant-preferred codons.

Recombination sites for use in the invention are known in the art and include FRT sites (see, for example, Schlake et al. (1994) Biochemistry 33:12746-12751; Huang et al. (1991) Nucleic Acids Res. 19:443-448; Sadowski (1995) Prog. Nuc. Acid Res. Mol. Bio. 51:53-91; Cox (1989) Mobile DNA, ed. Berg and Howe (American Society of Microbiology, Washington D.C.), pp. 116-670; Dixon et al. (1995) 18:449-458; Umlauf et al. (1988) EMBO J. 7:1845-1852; Buchholz et al. (1996) Nucleic Acids Res. 24:3118-3119; Kilby et al. (1993) Trends Genet. 9:413-421: Roseanne et al. (1995) Nat. Med. 1:592-594; Albert et al. (1995) Plant J. 7:649-659: Bailey et al. (1992) Plant Mol. Biol. 18:353-361; Odell et al. (1990) Mol. Gen. Genet. 223:369-378; and Dale et al. (1991) Proc. Natl. Acad. Sci. USA 88:10558-105620; all of which are herein incorporated by reference); and lox (Albert et al. (1995) Plant J. 7:649-659; Qui et al. (1994) Proc. Natl. Acad. Sci. USA 91:1706-1710; Stuurman et al. (1996) Plant Mol. Biol. 32:901-913; Odell et al. (1990) Mol. Gen. Genet. 223:369-378; Dale et al. (1990) Gene 91:79-85; and Bayley et al. (1992) Plant Mol. Biol. 18:353-361).

For purposes of the present invention, the DNA sequence encoding the recombinase protein and the target sequence for this recombinase protein can be derived from naturally occurring systems (as described above) either by being isolated from the relevant source by use of standard techniques or by being synthesized on the basis of known native sequences. Alternatively, variants or fragments of these DNA sequences may be used as long as they are capable of functioning in the intended manner. The functional variants or fragments may be prepared synthetically and may differ from the wild type sequence in one or more nucleotides.

The scorable marker genes and nucleotide sequences of interest coding for a desired product are constructed within the Mu transposable cassette as part of expression cassettes which allow for controlled expression in the organism of interest. The cassette will include 5′ and 3′ regulatory sequences operably linked to the scorable marker gene or nucleotide sequence of interest. By “operably linked” is intended a functional linkage between a promoter and a second sequence wherein the promoter sequence initiates and mediates transcription of the DNA sequence corresponding to the second sequence. Generally, “operably linked” means that the nucleic acid sequences being linked are contiguous and, where necessary to join two protein coding regions, contiguous and in the same reading frame. The cassette may additionally contain at least one additional gene to be transformed into the organism. Alternatively, the additional gene(s) can be provided on multiple expression cassettes or nucleotide fragments. Where both a scorable marker gene and a nucleotide sequence of interest are to be included in the Mu transposable cassette, the sequences can be assembled within the same expression cassette or within different expression cassettes. Such expression cassettes are provided with a plurality of restriction sites such that the inserted nucleotide sequences will be under the transcriptional regulation of the appropriate regulatory regions.

The expression cassette comprises in the 5′-3′ direction of transcription, a transcriptional and translational initiation region, a scorable marker gene or nucleotide sequence of interest, and a transcriptional and translational termination region functional in the host organism of interest. The transcriptional initiation region, the promoter, may be native or analogous or foreign or heterologous to the host. Additionally, the promoter may be the natural sequence or alternatively the promoter may be a synthetic sequence. By “foreign” is intended that the transcriptional initiation region is not found in the native organism into which the transcriptional initiation region is introduced.

While it may be preferable to express a nucleotide coding sequence using heterologous promoters, the native promoter sequences may be used. Such constructs would change expression levels of the encoded protein in the host organism, thereby altering its phenotype.

A number of promoters can be used in the practice of the invention. The promoters can be selected based on the desired outcome. Thus, the scorable marker gene sequences or nucleotide sequence of interest for coding for a desired polypeptide product can be combined with constitutive, inducible, tissue-preferred, or other promoters for expression in the organism of interest. Such promoters are well known in the art. Any promoter that is functional within the host organism can be used to drive expression of the coding sequence for the scorable marker gene or other nucleotide sequence of interest that comprises a coding sequence for a desired polypeptide product.

For example, where the organism is a plant, useful constitutive promoters include, but are not limited to, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; and 5,608,142.

Inducible promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also the copending application entitled “Inducible Maize Promoters,” U.S. application Ser. No. 09/257,583, filed Feb. 25, 1999, herein incorporated by reference. Other inducible promoters that are expressed locally at or near the site of pathogen infection, including, for example, those described in Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Chemical-regulated promoters can be used to modulate the expression of a gene through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression of a coding sequence within a particular tissue. Tissue-preferred promoters operable in plants, for example, include those described in Yamamoto et al. (1997) Plant J. 12(2)255-265; Kawamata et al. (1997) Plant Cell Physiol. 38(7):792-803; Hansen et al. (1997) Mol. Gen Genet. 254(3):337-343; Russell etal. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where the transformed organism is a plant, the gene(s) may be optimized for increased expression. That is, the genes can be synthesized using plant-preferred codons for improved expression. See, for example, Campbell and Gowri (1990) Plant Physiol. 92:1 -11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picomavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, N.Y.), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Thus, the integration vectors of the invention are engineered to comprise a nucleotide sequence of interest which may be stably integrated into the genome of a host organism. In this manner, the present invention provides a method for creating or enhancing the expression of a nucleotide sequence of interest in a host organism, or altering expression of an endogenous gene via antisense or sense suppression. Such an endogenous gene may be a naturally occurring host gene or a transgene previously integrated within the host organism.

Stable integration of a nucleotide sequence of interest into a host organism's genome is achieved by providing to the host organism an integration vector of the invention that comprises the sequence of interest and MuB protein. MuB protein may be provided as a purified protein or indirectly, via the MuB gene operably linked to a promoter that drives expression of a coding sequence in the host cell. Although the host organism can be transformed using separate transformation events for the integration vector and the plasmid carrying the MuB gene, these DNA constructs may be co-transformed into the host organism by a single transformation event. By “single” transformation event is intended that both of these DNA constructs are introduced into one or more cells of the host organism in the absence of an intervening selection step.

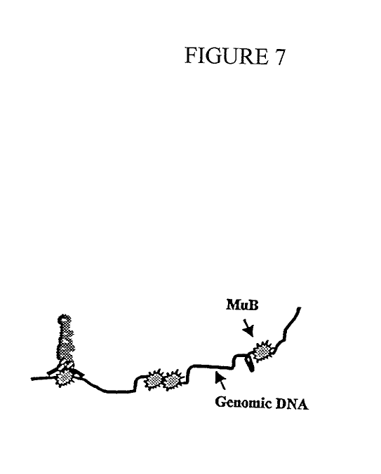

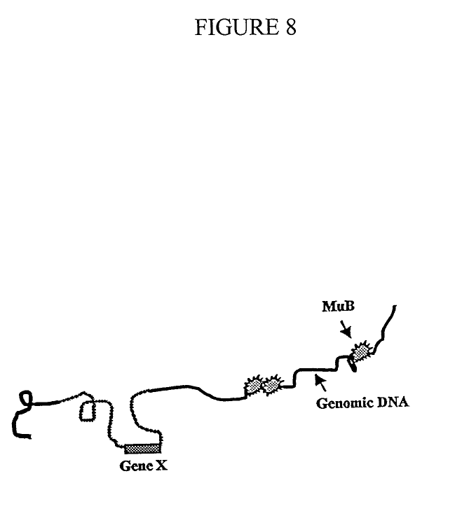

In the host cell, MuB binds to DNA of the host organism's genome in a random manner (see FIG. 6). The MuB-bound DNA then serves as a site for interaction between the MuA transposase within the MuA tetrameric core of the integration vector and the host DNA (see FIG. 7). The MuA transposase may then facilitate transfer of the Mu transposable cassette, and hence the nucleotide sequence of interest, into the host DNA at the site where MuB is bound (see FIG. 8). In addition to its role in bringing together the MuA transposase bound as part of the integration vector and the host DNA, MuB enhances the MuA transposase-driven transfer of DNA into the host genome.

The transformation methods of the present invention offer advantages over previous transformation methods that rely on direct delivery of naked DNA constructs or on the use of transposons. Direct delivery of DNA constructs into cells relies upon host cellular recombination mechanisms and involves host proteins that may not recognize the introduced DNA. In contrast, the transformation method of the present invention facilitates genetic recombination between the host genome and the introduced foreign DNA by providing an integration system to the host organism. In this way, the current invention eliminates the dependence of stable integration on host cellular recombination proteins. Furthermore, with the transformation method of the invention, the MuA transposase activity is achieved in the absence of in vivo expression of this protein within the host organism. Thus, the requirement for a transposase gene is eliminated. In this way, the current invention offers an advantage over other transposon-based transformation methods.

The gene-targeting methods of the invention can be utilized to genetically modify any organism of choice. By organism of choice is intended prokaryotic organisms, such as Escherichia coli, Bacillus subtilis, Pseudomonas species, etc., or eukaryotic organisms, including yeast, fungi, mammal, and more particularly plant species.

The present invention may be used for transformation of any plant species, including, but not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In some embodiments, plants of the present invention are crop plants (for example, corn, alfalfa, sunflower, Brassica, soybean, cotton, safflower, peanut, sorghum, wheat, millet, tobacco, etc.). In some embodiments, plants of the present invention are corn and soybean plants.

Methods for introducing constructs comprising DNA into prokaryotic or eukaryotic host cells are well known in the art. Transformation of a cell with DNA requires that the DNA be physically placed within the host cell. Current transformation procedures utilize a variety of techniques to introduce DNA into a cell. These include, but are not limited to, calcium phosphate transfection, DEAE-dextran-mediated transfection, cationic lipid-mediated transfection, electroporation, transduction, infection, lipofection, protoplast fusion, microparticle bombardment, and other techniques such as those found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989). In one form of transformation, the DNA is microinjected directly into cells though the use of micropipettes. Alternatively, high velocity ballistics (such as, for example, those deliverable with the use of a “gene gun” or the like) is used to introduce DNA and associated molecules and proteins (e.g., spermidine) into the cell. In another form, the cell is permeabilized by the presence of polyethylene glycol, thus allowing DNA and other molecules to enter the cell through diffusion. DNA can also be introduced into a cell by fusing protoplasts with other entities which contain DNA. These entities include minicells, cells, lysosomes or other fusible lipid-surfaced bodies. Electroporation is also an accepted method for introducing DNA solutions into a cell. In this technique, cells are subject to electrical impulses of high field strength which reversibly permeabilizes biomembranes, allowing the entry of exogenous DNA solutions. Any such method for directly introducing DNA into a prokaryotic or eukaryotic cell can be used with the novel integration vectors of the present invention to obtain transformed cells comprising the Mu transposable element integrated within a predetermined target site in the host organism's genome.

Thus, in one embodiment of the invention, the disclosed integration vectors of the present invention are introduced into the nucleus of a plant cell by any method available in the art. In this manner, genetically modified plants, plant cells, plant tissue, seed, and the like can be obtained. These constructs may be introduced into the plant by one or more techniques typically used for direct DNA delivery into cells. Such protocols may vary depending on the type of plant or plant cell, i.e., monocot or dicot, targeted for gene modification. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice). In one embodiment, the integration vector of the invention and the plasmid comprising the MuB gene are co-transformed into a plant using particle bombardment.

The plant cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

The following examples are offered by way of illustration and not by way of limitation.

### EXPERIIMENTAL

### Example 1

### Construction of a Mini-Mu Plasmid

A plasmid PC-4 comprising a gene of interest operably linked to a promoter within the Mu transposable cassette was constructed as shown in FIG. 9. The ends of the Mu transposable cassette were defined by the MuA recognition and binding sites R1, R2, and R3 arranged in inverted repeat configuration. The R1, R2, and R3 sites may be variants of reported sequences, so long as MuA is capable of binding to the sites so as to form an active CDC capable of stable integration into the host genome. In plasmid PC-4, the Mu transposable cassette carried the selectable market gene CM(R), which confers resistance to chloramphenicol. Plasmids PC1, PC-2, and PC-3 were also constructed, as shown in FIG. 10. In each of these plasmids, as in plasmid PC-4, the ends of the Mu transposable cassette were defined by the MuA recognition and binding sites R1, R2, and R3 in inverted repeat configuration. Each of plasmids PC1, PC-2 and PC-3 contained the ampicillin gene in the non-Mu plasmid domain, or region of the plasmid outside the Mu transposable cassette. Within the Mu transposable cassette, the PC-1 plasmid contained an internal activating sequence or IAS; the PC-2 plasmid contained CM(R) or chloramphenicol resistance gene and an internal activating sequence, and the PC-3 plasmid contained the CM(R) chloramphenicol resistance gene.

### Example 2

### Transformation and Regeneration of Transgenic Plants

Immature maize embryos from greenhouse donor plants are co-bombarded with a plasmid containing the MuB gene operably linked to the ubiquitin promoter and the integration vector of example 1, which comprises the gene of interest operably linked to the ubiquitin promoter. Transformation is performed as follows. Media recipes follow below.

Preparation of Target Tissue

The ears are surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

The integration vector and plasmid comprising the MuB gene are precipitated onto 1.1 μm (average diameter) tungsten pellets using a CaCl₂ precipitation procedure as follows:

100 μl prepared tungsten particles in water

10 μl (1 μg) DNA in TrisEDTA buffer (1 μg total)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J. medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for expression of the nucleotide sequence of interest.

Bombardment and Culture Media

Bombardment medium (560Y) comprises 4.0 g/l N6 basal salts (SIGMA C-1416), 1.0 ml/l Eriksson's Vitamin Mix (1000X SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000X SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos(both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 3

### Soybean Embryo Transformation

Soybean embryos are bombarded with an active CDC as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with fluorescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 32 7:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for stably integrating a nucleotide sequence of interest into the genome of a host organism, said method comprising:
a) providing MuB protein within at least one cell of said host organism; and
b) providing said at least one cell of said host organism with an integration vector comprising a Mu transposable cassette derived from a mini-Mu plasmid, wherein said Mu transposable cassette comprises said nucleotide sequence of interest, and wherein said Mu transposable cassette is bound to MuA transposase or biologically active variant thereof to form an active cleaved donor complex,
whereby said Mu transposable cassette comprising said nucleotide sequence of interest is stably integrated into the genome of said host organism.

2. The method of claim 1, wherein the mini-Mu plasmid from which said Mu transposable cassette is derived is a wild-type mini-Mu plasmid.

3. The method of claim 1, wherein the mini-Mu plasmid from which said Mu transposable cassette is derived is a derivative mini-Mu plasmid.

4. The method of claim 1, wherein MuB is provided within said at least one cell by providing a plasmid within said cell, said plasmid comprising a coding sequence for a MuB protein or biologically active variant thereof operably linked to a promoter that drives expression of a coding sequence in said host organism.

5. The method of claim 1, wherein said nucleotide sequence of interest is selected from the group consisting of an antisense sequence for a gene of interest, a sense sequence for a gene of interest, and a coding sequence for a polypeptide of interest, wherein said nucleotide sequence of interest is operably linked to a promoter that drives expression in said host organism.

6. The method of claim 5, wherein said Mu transposable cassette further comprises a scorable marker gene operably linked to a promoter that drives expression of said scorable marker gene in said organism of interest.

7. The method of claim 1, wherein said host organism is a plant.

8. The method of claim 7, wherein said plant is a monocot.

9. The method of claim 8, wherein said monocot is selected from the group consisting of maize, wheat, sorghum, rice, barley, oats, and rye.

10. The method of claim 7, wherein said plant is a dicot.

11. The method of claim 10, wherein said dicot is selected from the group consisting of soybean, sunflower, canola, cotton, alfalfa, potato, sugar beet, and safflower.

12. A method for stably integrating a nucleotide sequence of interest into the genome of a host organism, said method comprising the steps of:
a) transforming at least one cell of said organism with an integration vector comprising a Mu transposable cassette from a precleaved mini-Mu plasmid, wherein said Mu transposable cassette comprises said nucleotide sequence of interest, and wherein said Mu transposable cassette is bound to MuA transposase or biologically active variant thereof to form an active cleaved donor complex; and
b) providing MuB protein within said at least one cell,
whereby said Mu transposable cassette comprising said nucleotide sequence of interest is stably integrated into the genome of said host organism.

13. A host cell having a Mu transposable cassette stably integrated within its genome.

14. An integration vector comprising a Mu transposable cassette derived from a precleaved mini-Mu plasmid, wherein said Mu transposable cassette is bound to MuA transposase to form an active cleaved donor complex (CDC), and wherein said Mu transposable cassette comprises a nucleotide sequence of interest.

15. An integration vector comprising an active CDC, said active CDC comprising a Mu transposable cassette having a first end and a second end, and further comprising one or more MuA binding sites at each of said ends of the Mu transposable cassette, said MuA binding sites being selected from the group of: R1, R2, R3, L1, L2, and L3.
